(19) United States
(12) Patent Application Publication
(10) Pub. No.: US 2006/0246027 A1
(43) Pub. Date: Nov. 2, 2006

(54) Personal care composition

(76) Inventor: Paul Robert Tanner, Lebanon, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/120,027

(22) Filed: May 2, 2005

(51) Int. Cl. A61K 8/19; A61K 8/89

(52) U.S. Cl. 424/70.12; 424/401

(57) ABSTRACT

Stable personal care composition comprising hydrophobic modified silica, a particulate material and a dermatologically-acceptable carrier, said carrier comprising about 70% or greater, by weight of the carrier, of at least one water-soluble polyol, said water-soluble polyol having a solubility parameter of from about 11 to about 17.

### FIELD OF THE INVENTION

The present invention relates to a personal care composition useful, for example, as a cosmetic mask or peel. The present invention further relates to a method for improving the condition of mammalian keratinous tissue by applying the composition either alone or in conjunction with additional compositions.

### BACKGROUND OF THE INVENTION

A variety of products are available to the consumer to counteract what many consider undesirable “signs of skin aging,” such as fine lines, wrinkles and uneven skin texture. Cosmetic masks and peels are thought to help rejuvenate skin by, among other means, promoting exfoliation and providing skin conditioning benefits. Cosmetic masks and peels are available in a variety of forms, including clay-like compositions that typically are washed off after drying, compositions that are peeled off after drying, and compositions that are rinsed from the skin before drying.

Carriers for masks and peels may be either hydrophobic or hydrophilic. Hydrophobic carriers may contain, for example, mineral oils or silicone, whereas hydrophilic carriers may contain water or polyols. Hydrophilic carriers often are preferred because they are more easily rinsed from the skin. Particulate materials also are desirable for promoting exfoliation, improving skin feel, and/or providing desirable visual effects. In order to uniformly suspend the particulate material in the carrier, a suitable thickener must be used. Some commonly used thickeners include waxes, polymers, and hydrophilic silica. A drawback of using waxes is that heat is required to melt and recrystallize the wax, which can destabilize certain ingredients, such as vitamins and other actives, and particulates such as bicarbonates. Polymers, for example cellulosic polymers, tend to impart a greasy or heavy feel to the skin, which consumers may find unacceptable. Hydrophilic silica requires no heating and may have acceptable skin feel characteristics. However, Applicants unexpectedly have found that mask or peel compositions that utilize hydrophilic silica as the primary thickening ingredient can exhibit instability under typical use, transport, and storage conditions. For example, low levels of shear slight mixing, physical shock resulting from bumping or dropping the container, or an increase in temperature all may result in separation of the mask components. Particulate materials may temporarily or permanently cease to be suspended, and instead aggregate at the bottom of the container. This results in a product that is aesthetically unappealing to consumers, and which less effectively delivers the desired skin care benefits.

To overcome this instability problem, Applicants have sought alternative thickeners in which the particulate material remains substantially uniformly suspended. In addition, compositions containing the thickener must exhibit properties that are pleasing to the consumer, such as effective conditioning, exfoliation, good rinsing and clean feel.

There exists a need, therefore, for personal care compositions that are stable under reasonable use and storage conditions, that more uniformly suspend particulate materials, that require no heating during manufacture, and which exhibit properties pleasing to the consumer.

### SUMMARY OF THE INVENTION

The present invention meets the aforementioned need. Applicants have found that the use of hydrophobic modified silica as a thickener meets all of the requisite criteria. This was unexpected, because hydrophobic compounds tend to adhere to the surface of skin and other keratinous tissue, and as such, one of skill in the art would not expect hydrophobic modified silica to exhibit good rinsing characteristics. In addition, Applicants have found that hydrophobic modified silica effectively suspends particulate materials, and thus produces compositions that are stable under typical use, transport and storage conditions. Because hydrophobic silica requires no heating during the manufacturing process, heat-sensitive actives and particulates, such as bicarbonate salts, can be included in the compositions. Bicarbonate salts and other water-soluble particulate materials have the advantages of good rinsing characteristics, and also at least partial, if not complete, dissolution upon rinsing. This limits gritty residue left on the skin and messiness associated with use. Furthermore, compositions comprising bicarbonate salts can be used together with an acid-containing composition, which results in effervescence. This provides an immediate indication to the consumer that the compositions are having their intended effect.

The use of water-soluble particulates raises additional considerations in choosing a suitable carrier for the composition. To have good rinsing characteristics, the carrier must be water soluble. To stabilize the water-soluble particulates, the carrier must have limited water content or be substantially anhydrous. Applicants have found that a suitable carrier in the compositions of the present invention is one which comprises a high percentage of one or more polyols having a solubility parameter of from about 11 to about 17. Additionally, Applicants have found that when such a carrier is substantially anhydrous, upon rinsing with water a warming sensation may be produced. Without being limited by theory, Applicants believe that the water reacts exothermically with the polyol. Besides being pleasing to the consumer, the warming may have the additional advantage of increasing circulation to the keratinous tissue. This in turn may result in, for example, more efficient delivery of nutrients to the tissue, and a more healthy appearance, or “glow,” which may be especially desirable in the facial area.

According to the first embodiment of the present invention, a stable personal care composition is provided, comprising hydrophobic modified silica, a particulate material, and a hydrophilic carrier, said carrier comprising about 70% or greater, by weight of the carrier, of one or more suitable water-soluble polyols. The particulate material remains substantially uniformly suspended throughout the exfoliating composition under reasonable transport, storage and use conditions. The particulate material may be water soluble, and substantially dissolve upon rinsing.

Yet another embodiment of the present invention provides for a personal care composition according to the first embodiment, wherein said composition further comprises one or more skin care actives.

In yet another embodiment of the present invention, a personal care composition according to either embodiment is releasably deposited onto a substrate.

Yet another embodiment of the present invention provides a method for regulating the condition of mammalian keratinous tissue, comprising the step of applying to the keratinous tissue a first composition comprising hydrophobic modified silica, a particulate material, and a hydrophilic carrier, said carrier comprising about 70% or greater, by weight of the carrier, of one or more suitable water-soluble polyols. The particulate material remains substantially uniformly suspended throughout the exfoliating composition under reasonable transport, storage and use conditions. The particulate material may be water soluble, and may partially or completely dissolve upon rinsing. When the carrier is substantially anhydrous, heat may be produced upon rinsing with water.

In yet another embodiment of the present invention, a method for regulating the condition of mammalian keratinous tissue is provided, comprising the steps of applying the aforementioned first composition to the keratinous tissue, and the step of applying a second composition comprising an acidic compound such that the second composition contacts, or commingles with, the first composition. The first and second compositions may comprise one or more skin care actives. In one embodiment, the particulate material in the first composition is a bicarbonate salt, whereupon application of the second composition may result in effervescence.

In yet another embodiment of the present invention, a method for regulating the condition of mammalian keratinous tissue is provided, comprising the steps of applying a first composition described herein to the keratinous tissue, and the step of applying one or more additional compositions to the keratinous tissue. The additional composition(s) may comprise one or more skin care actives. Optionally, a second composition comprising an acidic compound may be applied such that the second composition contacts, or commingles with, the first composition.

In yet another embodiment of the present invention, a method for regulating the condition of mammalian keratinous tissue is provided comprising the steps of applying a first composition described herein to the keratinous tissue, and orally ingesting a composition comprising one or more dietary supplements. Optionally, a second composition as described herein also may be applied such that the second composition contacts, or commingles with, the first composition.

Yet another embodiment provides for a kit, said kit comprising a first composition as described herein and a second composition as described herein.

Yet another embodiment provides for a kit, said kit comprising a first composition as described herein, and one or more additional compositions. The kit further may comprise a second composition as described herein, and one or more dietary supplements.

### DETAILED DESCRIPTION OF THE INVENTION

Whereas the specification concludes with claims that particularly point out and distinctly claim the present invention, it is believed that the invention will be better understood from the following details.

The present invention describes personal care compositions suitable for use, for example, as a cosmetic mask. The compositions comprise hydrophobic modified silica, a particulate material, and a hydrophilic carrier, said carrier comprising about 70% or greater, by weight of the carrier, of a water-soluble polyol. The polyol has a solubility parameter of from about 11 to about 17. When used to describe embodiments that comprise more than one step or component, such as a method or a kit, the compositions of the present invention so described may be referred to as a “first composition.”

The compositions of the present invention may have a viscosity of from about 1,000 cps to about 10,000,000 cps, and alternatively from about 10,000 cps to about 5,000,000 cps, where “cps” means centipoise and is measured under ambient conditions. Alternatively, the compositions may range from transparent, to translucent, to opaque. The compositions may be used in cosmetics, hair care and skin care products, non-limiting uses of which include cleansers, moisturizers, conditioners, anti-aging compounds, exfoliants, peels, and combinations thereof. In one embodiment, the compositions are applied to the face, neck and other exposed areas of the body.

When the carrier of the composition of the present invention is substantially anhydrous, upon rinsing with water heat may be produced. The amount of heat produced is sufficient to provide a pleasant warming sensation, yet does not result in undue discomfort or other undesirable effects. Alternatively, heat may be produced upon application of a second and/or additional composition that comprises water. Alternatively, some heat may be produced upon application to the keratinous tissue, without the tissue being rinsed. Alternatively, heat may be produced when the composition of the present invention comprises a sulfate salt, for example, magnesium sulfate, and/or a chloride salt, for example, calcium chloride.

When the compositions of the present invention comprise a water-soluble particulate material, the particulate may substantially dissolve upon rinsing with water.

When a first composition of the present invention comprising a bicarbonate salt is applied to the keratinous tissue, upon application of a second composition comprising an acidic compound, effervescence may occur.

The compositions further may be releasably applied to a carrier substrate, suitable for use at a later time. The compositions further may be used in combination with a delivery enhancement device, non-limiting examples of which include an implement, a spray applicator, a brush, an automated scrubbing device, an ultrasound device, and combinations thereof. The compositions further may be used in conjunction with orally ingestible dietary supplement to provide enhanced skin care benefits.

The compositions of the present invention may be removed through a variety of means, for example, through rinsing the skin with water.

Each of the above and additional elements is described herein.

In all embodiments of the present invention, all percentages are by weight of the total composition, unless specifically stated otherwise. All ratios are weight ratios, unless specifically stated otherwise. The number of significant digits conveys neither limitations on the indicated amounts nor on the accuracy of the measurements. All amounts indicating quantities, percentages, proportions, etc. are understood to be modified by the word “about” unless otherwise specifically indicated. All measurements are understood to be made at about 25° C. and at ambient conditions, where “ambient conditions” means conditions under about one atmosphere of pressure and at about 50% relative humidity.

Herein, “stable” and “stability” mean compositions which are substantially unaltered in chemical state, physical homogeneity and/or color upon exposure to conditions reasonably expected to be incurred in shipping, storage and use. Stability may be determined either by empirical observation or by appropriate methods of chemical and/or physical analysis that would be known to one of skill in the art. “Stable” and “stability” are understood to mean that the particulate material remains substantially uniformly suspended in the composition.

Herein, “substantially uniformly suspended” means that there is no clear or transparent layer visibly present that contains a lower concentration of particulates than an opaque layer that contains significant amounts of particulate material. Alternatively, in the concentration of particulate material in various layers of the composition may be determined by means of chemical and/or physical analysis. This may be especially useful when the particulate material is sufficiently small so as to result in translucent or transparent layers that are not readily discernable.

Herein, “substantially anhydrous” means that water is not added to the carrier; however, small amounts of water incidentally may be present, for example as a by-product of chemical reactions, or as a result of absorption from the environment.

Herein, “reasonable shipping, storage and use conditions” means reasonable commercial and consumer storage conditions, reasonable transport conditions, and conditions reasonably expected to be incurred during consumer use. Such conditions are understood to include, but are not limited to: physical shock, for example, shaking, dropping, bumping; friction, for example, touching, stirring; and temperature changes, including commercial transport and commercial storage conditions with exposure to temperatures of from about −20° C. to about 60° C.

Herein, “first composition” means a composition comprising hydrophobic modified silica, a particulate material, and a carrier comprising one or more water-soluble polyols. Herein, “second composition” means a composition comprising an effective amount of an acidic compound, for example, lactic acid, and additionally may comprise any of the other components described herein. Herein, “additional composition” means a composition that may comprise any of the elements of the first and second compositions described herein. The second and additional compositions may be in a variety of forms, non-limiting examples of which include a lotion, cream, paste, milk, liquid, gel, solid, spray, mousse, eye jelly, mask, and combinations thereof. The second and additional compositions may be in the form of an emulsion, and may impart a wide variety of benefits to the skin or other keratinous tissue, including regulating and improving the condition of the skin.

Herein, “mask” means a composition that is applied to the keratinous tissue, for example to the face and/or neck. The mask is allowed to remain on the keratinous tissue for a period of time suitable to exert a desired effect, non-limiting examples of which include drying, tightening, pore-size reduction, moisturizing, exfoliating, heating, foaming, delivery of actives, etc. The mask may be removed, for example, by wiping, rinsing with water, scrubbing, peeling, etc. The mask composition further may be applied to a substrate which is worn on the desired area of keratinous tissue for a suitable period of time.

“Keratinous tissue,” as used herein, means keratin-containing layers disposed as the outermost protective covering of mammals and includes, but is not limited to, skin, hair and nails. “Topical application,” as used herein, means to apply or spread a composition onto the surface of the keratinous tissue.

Herein, “personal care composition” means compositions suitable for topical application on mammalian keratinous tissue. The personal care compositions described herein may contain one or more skin care actives. “Skin care actives,” or “actives,” as used herein, means compounds that, when applied to the skin, hair or other keratinous tissue, provide a benefit and/or improvement to the keratinous tissue.

Herein, “skin care” means regulating and/or improving skin condition. Herein, “regulating skin condition” means effecting desirable skin appearance and/or feel, for example, by providing a smoother appearance and/or feel. Herein, “improving skin condition” means effecting a visually and/or tactilely perceptible positive change in skin appearance and feel. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, “signs of skin aging,” include, but are not limited to, outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; flaking; dryness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Dermatologically-acceptable,” as used herein, means that the compositions or components thereof so described are suitable for use in contact with mammalian keratinous tissue without undue toxicity, incompatibility, instability, allergic response, and the like.

Herein, “orally acceptable” means that the compositions or components thereof so described are suitable for oral ingestion by a mammal without undue toxicity, incompatibility, instability, allergic response, and the like.

“Effective amount,” as used herein, means an amount of a compound or composition sufficient to significantly induce a positive benefit, including independently or in combination the benefits disclosed herein, but low enough to avoid serious side effects.

Herein, “delivery enhancement device” means any device that increases the amount of composition applied to and/or into the skin, more easily and/or efficiently delivers the composition, and/or increases the beneficial results derived from the composition, relative to that delivered without using the device.

Herein, “dietary supplement” means a dietary ingredient intended to supplement a regular diet, non-limiting examples of which include vitamins, minerals, herbs or other botanicals, amino acids, enzymes and metabolites. Herein, the dietary supplement is suitable for oral consumption and is administered orally. Examples of dietary supplements suitable for use in the present invention include, but are not limited to, vitamins and vitamin derivatives, peptides, essential fatty acids, and sugar amines. The form in which the dietary supplement is administered may vary widely, and includes, for example, tablets, capsules, gel tablets, and liquids. The dietary supplement further may be incorporated into a foodstuff or beverage.

Herein “kit” means a packaging unit comprising at least one composition described herein. The kit may comprise an outer packaging unit, which in turn may comprise one or more inner packaging units. The inner and outer packaging units may be of any type suitable for containing, presenting and/or reasonably protecting from damage the contents of the kit. The kit may comprise a plurality of components, such as a first composition and a second composition, as described herein. The kit further may comprise one or more additional compositions, one or more orally ingestible dietary supplements, a delivery enhancement device, instructions for use of the device, instructions for complying with suitable application regimens, a substrate, and combinations thereof.

I. Hydrophobic Modified Silica

Herein, hydrophobic modified silica means one or more fumed hydrophobic silica compounds. Fumed silica is produced in a hydrogen gas flame according to the reaction SiCl₄+2H₂+2O₂→SiO₂+4HCl. Untreated fumed silica contains hydrophilic surface silanol and siloxane groups. Hydrophobic fumed silica is formed by chemical reaction of the surface silanol and siloxane groups with hydrophobic modifying compounds. Suitable hydrophobic modifying compounds include, but are not limited to, dimethyl silyl, trimethylsiloxyl and dimethicone, to produce respectively silica dimethyl silylate, silica silylate, and silica dimethicone silylate. Fumed silicas are described by S. Hasenzahl and A. Braunagel in “Fumed silica for personal care and cosmetics—versatile and effective,” SOFW-Journal, vol. 129, issue 8 (2003), p. 2-8.

Suitable hydrophobic modified silicas include, but are not limited to, the Aerosil R series (wherein “R” means “repellent”), for example, Aerosil R805, R812, R972, R974, R976, R976 S, R8200, RX200 and RX300, available from Degussa AG; Cab-o-sil TS-530, TS-610, TS-720, available from Cabot Corp.; and HDK H2000, H15, H18, H₂O and H30, available from Wacker-chemie GmbH, and mixtures thereof. In one embodiment, the hydrophobic modified silica of the present invention is selected from the group consisting of silica dimethyl silylate, silica silylate, silica dimethicone silylate, and combinations thereof. Alternatively, the hydrophobic modified silica is selected from the group consisting of silica silylate, silica dimethicone silylate and combinations thereof. Alternatively, the hydrophobic modified silica has a carbon content of about 1.5% or greater, by weight of the hydrophobically-treated fumed silica. Alternatively, the hydrophobic modified silica has a carbon content of about 2.0% or greater, by weight of the hydrophobically-treated fumed silica.

The compositions of the present invention may include from about 1% to about 15%, alternatively from about 2% to about 10%, alternatively from about 3% to about 8% of the hydrophobic modified silica of the present invention.

II. Particulate Material

The compositions of the present invention may comprise from about 1% to about 50%, alternatively from about 5% to about 40%, and alternatively from about 10% to about 40%, of a particulate material including, but not limited to, cleansing agents, exfoliating agents, skin feel modifiers, and optical modifiers. The particulate material may range in size from about 0.1 microns to about 5,000 microns, and alternatively from about 0.25 microns to about 1000 microns. In one embodiment, all or part of the particulate material substantially dissolves upon rinsing with water. In another embodiment, the particulate material effervesces when placed in contact with a composition comprising an acidic component. In yet another embodiment, when the particulate material is a sulfate salt, for example, magnesium sulfate, and/or a chloride salt, for example, calcium chloride, heat may be generated when the composition is rinsed with water, or is placed in contact with a composition comprising water.

Alternatively, the particulate material is an inorganic salt. Alternatively, the particulate material is selected from the group consisting of sodium bicarbonate, magnesium sulfate, sodium chloride, calcium chloride, and mixtures thereof. Alternatively, the particulate material is a bicarbonate salt. Alternatively, the particulate material is sodium bicarbonate.

Non-limiting examples of suitable particulate materials can be found in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2728. Suitable particulate materials include, but are not limited to almond meal, aluminum oxide, apricot seed powder, bismuth oxychloride, boron nitride, cellulose and cellulose derivatives, clay, calcium oxide, iron oxide, jojoba seed powder, loofah, mica, peach pit powder, pecan shell powder, polyethylene, polybutylene, polyisobutylene, polymethylstyrene, polypropylene, polystyrene, polyurethane, nylon, polytetrafluoroethylene, polyhalogenated olefins, pumice, rice bran, sericite, silk, synthetic hectorite, titanium dioxide, tricalcium phosphate, and mixtures thereof. Also useful are particles made from mixed polymers (e.g., copolymers, terpolymers, etc.), among such are polyethylene/polypropylene copolymer, polyethylene/propylene/isobutylene copolymer, polyethylene/styrene copolymer, and mixtures thereof. Typically, the polymeric and mixed polymeric particles are treated via an oxidation process, for example to destroy impurities. The polymeric and mixed polymeric particles can also optionally be cross linked with a variety of common crosslinking agents, non-limiting examples including butadiene, divinyl benzene, methylenebisacrylamide, allyl ethers of sucrose, allyl ethers of pentaerythritol, and mixtures thereof. Other examples of useful particles include waxes and resins such as paraffins, carnuba wax, ozekerite wax, candellila wax, and urea-formaldehyde resins. When such waxes and resins are used herein it is important that these materials are solids at ambient and skin temperatures.

Other examples of particulate materials useful in the present invention include colored and uncolored pigments, interference pigments, inorganic powders and organic powders other than those described above, composite powders, optical brightener particles, and mixtures thereof. The average size of such particulates in general may be smaller than the aforementioned particulate materials, ranging for example from about 0.1 microns to about 100 microns. These particulates can, for example, be platelet shaped, spherical, elongated or needle-shaped, or irregularly shaped, surface coated or uncoated, porous or non-porous, charged or uncharged, and can be added to the current compositions as a powder or as a pre-dispersion. These particulate materials can be derived from natural and/or synthetic sources.

Suitable organic powders particulate materials include, but are not limited, to polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); sphericle particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminium starch octenylsuccinate, for example Dry Flo™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

Also useful herein are interference pigments. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃. Such pigments often are pearlescent. Pearlescent pigments reflect, refract and transmit light because of the transparency of pigment particles and the large difference in the refractive index of mica platelets and, for example, the titanium dioxide coating. Intereference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Other pigments useful in the present invention can provide color primarily through selective absorption of specific wavelengths of visible light, and include inorganic pigments, organic pigments and combinations thereof. Examples of such useful inorganic pigments include iron oxides, ferric ammonium ferrocyanide, manganese violet, ultramarine blue, and chromium oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO₂, ZnO, or ZrO₂, are commercially available from a number of sources, for example, TRONOX TiO₂ series, SAT-T CR837, a rutile TiO2 (U.S. Cosmetics). Also suitable are charged dispersions of titanium dioxide, disclosed in U.S. Pat. No. 5,997,887, issued to Ha et al.

The pigments and/or powders of the current invention may be surface treated to provide added stability of color and/or for ease of formulation. Non-limiting examples of suitable coating materials include silicones, lecithin, amino acids, metal soaps, polyethylene, collagen, and combinations thereof.

III. Carrier

The compositions of the present invention comprise from about 35% to about 98%, alternatively from about 50% to about 93%, and alternatively from about 52% to about 87%, by weight of the composition, of a carrier. The carrier may comprise about 10% or less, alternatively about 5% or less, and alternatively about 2% or less, by weight of the carrier, of water. Alternatively, the carrier may be substantially anhydrous.

1. Water-Soluble Polyol

The carrier comprises about 70% or greater, by weight of the carrier, of one or more water-soluble polyols. Alternatively, the carrier may comprise about 80% or greater, and alternatively about 90% or greater, of one or more water-soluble polyols. Herein, “water soluble polyol” is understood to include isomers, salts and derivatives of the polyol. Polyols suitable for use herein include any natural and synthetic polyol with an overall solubility parameter of from about 11 to about 17.

Solubility parameters for the polyols described herein are determined by methods known to one skilled in the chemical arts, and are expressed in units of (cal per cm³)^{1/2}, where “cal” means calories and “cm³” means cubic centimeters. Solubility parameters are discussed extensively by C. D. Vaughan in “The Solubility Parameter: What is it?,” Cosmetics & Toiletries, vol. 106, November, 1991, pp. 69-72, and also by C. D. Vaughan in “Using Solubility Parameters in Cosmetics Formulation”, 36 J. Soc. Cosmetic Chemists 319-333, September/October, 1988.

Examples of suitable polyols and polyol derivatives include, but are not limited to, glycerol, diglycerol, triglycerol, ethylene glycol, propylene glycol, dipropylene glycol, tripropylene glycol, butylene glycol, pentylene glycol, hexylene glycol, erythritol, 1,2,6-hexanetriol, polyethylene glycols, such as PEG-4, PEG-5, PEG-6, PEG-7, PEG-8, PEG-9, PEG-10, PEG-11, PEG-12, PEG-14, PEG-16, PEG-18, PEG-20, PEG-32, PEG-40, PEG-55, PEG-60, PEG-75, PEG-90, PEG-100); sugars and sugar derivatives, such as fructose, glucose, maltose, maltitol, mannitol, inositol, sorbitol, sorbityl silanediol, sucrose, trehalose, xylose, xylitol, glucuronic acid; sorbitol, ethoxylated sorbitol, and mixtures thereof.

Alternatively, the water-soluble polyol is selected from the group consisting of glycerol, propylene glycol, dipropylene glycol, tripropylene glycol, butylene glycol, pentylene glycol, hexylene glycol, polyethylene glycol, and combinations thereof. Alternatively, the water-soluble polyol is a polyethylene glycol. Alternatively, the water-soluble polyol is a polyethylene glycol selected from the group consisting of PEG-4, PEG-5, PEG-6, PEG-7, PEG-8, PEG-9, PEG-10, PEG-11, PEG-12, and mixtures thereof.

2. Optional Ingredients

The compositions of the present invention may comprise one or more of the following optional ingredients.

a. Surfactants

The compositions of the present invention may comprise one or more surfactants, useful for generating foam upon rinsing with water and/or upon application of a second and/or additional composition. These surfactants or combinations of surfactants should be mild, which means that these surfactants provide sufficient cleansing or detersive benefits but do not overly dry the skin. Surfactants useful herein include those selected from the group consisting of anionic surfactants, amphoteric surfactants, zwitterionic surfactants, cationic surfactants, nonionic surfactants and mixtures thereof. In one embodiment, the surfactant is a non-ionic surfactant. Examples of such surfactants are found in and U.S. Pat. No. 5,624,666, issued to Coffindaffer et al. Concentrations of these surfactants are from about 0.1% to about 20%, alternatively from about 0.5% to about 15%, and alternatively from about 1% to about 10%, by weight of the carrier.

b. Polymeric Thickening Agent

The compositions of the present invention may comprise from about 0.1% to about 5%, alternatively from about 0.1% to about 4%, and alternatively from about 0.25% to about 3%, by weight of the carrier, of one or more polymeric thickening agents, including polymeric thickeners and gelling agents. Nonlimiting classes of thickening agents include crosslinked polyacrylate polymers, polyacrylamide polymers, polysaccharides and gums. In one embodiment, the polymeric thickening agent is a cellulose derivative.

c. Substrates

The compositions of the present invention can be applied directly to the skin. Additionally or alternatively, the compositions can be applied with the use of a suitable applicator comprising a substrate material. In one embodiment, the composition is applied to the substrate such that the substrate releasably holds the composition. In one embodiment, the composition is combined with or deposited onto the substrate prior to packaging. The substrate combined with the composition may be packaged in a relatively dry state, and wetted prior to use, or may be packaged having already been wetted.

Suitable substrates include, but are not limited to, nonwovens, films, foams, sponges, and combinations thereof. In one embodiment, wipe substrates comprise a porous material which is capable of holding the composition within the pores of the substrate. In one embodiment, the substrate is a nonwoven.

Techniques for combining wipe substrates with a cleansing or treating composition, and for their packaging, are well known in the art and are applicable to the present invention. In general, the substrate is combined with the composition by one or more techniques involving coating, immersing, dipping, spraying, extruding. In general, the substrates are combined with an amount of the composition sufficient to provide effective skin application. In one embodiment, the product may be combined with the substrate in amounts of from about 0.1 gram of lotion per gram of substrate to about 10 grams of lotion per gram of substrate.

d. Skin Care Actives

The compositions of the present invention may comprise at least one additional skin care active, useful for regulating and/or improving the condition of mammalian skin and other keratinous tissue. Classes of suitable skin care actives include, but are not limited to vitamins, peptides and peptide derivatives, sugar amines, sunscreens, oil control agents, flavonoid compounds, hair growth regulators, antioxidants and/or preservatives, phytosterols, protease inhibitors, tyrosinase inhibitors, anti-inflammatory agents, and mixtures thereof. It should be noted, however, that many skin care actives may provide more than one benefit, or operate via more than one mode of action. Therefore, classifications herein are made for the sake of convenience and are not intended to limit the active to that particular application or applications listed.

i. Vitamins

The compositions of the present invention may comprise one or more vitamins, for example, to provide antioxidant and/or other nutritional benefits to the keratinous tissue. Herein, “vitamins” means vitamins, pro-vitamins, and their salts, isomers and derivatives. The vitamins may include water soluble vitamins, for example, vitamin B compounds (including niacinamide, nicotinic acid, C1-C18 nicotinic acid esters, and nicotinyl alcohol; B6 compounds, such as pyroxidine; and B5 compounds, such as panthenol, or “pro-B5”); and vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate; and mixtures thereof. The vitamins also may include those exhibiting limited solubility in water, such as vitamin A compounds, and all natural and/or synthetic analogs of Vitamin A, including retinoids, carotenoids, and other compounds which possess the biological activity of Vitamin A; vitamin D compounds; vitamin E compounds, or tocopherol, including tocopherol sorbate, tocopherol acetate, other esters of tocopherol; vitamin K compounds; and mixtures thereof. In one embodiment, the compositions of the instant invention may comprise from about 0.0001% to about 10%, alternatively from about 0.001% to about 8%, alternatively from about 0.01% to about 5%, and alternatively from about 0.1% to about 1%, of the vitamin.

ii. Peptides and Peptide Derivatives

The compositions of the present invention may comprise one or more peptides, for example, to aid in repair of keratinous tissue, to aid in exfoliation, and to deliver other benefits to the keratinous tissue. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

iii. Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-D-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

iv. Sunscreens

The compositions of the present invention may comprise one or more sunscreen actives (or sunscreen agents) and/or ultraviolet light absorbers, and may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives are 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

In one embodiment, the composition may comprise from about 1% to about 30%, and alternatively from about 2% to about 20% by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

v. Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.1% to about 2%, of an oil control agent.

vi. Flavonoids

The compositions of the present invention may comprise a flavonoid, for example, to provide anti-oxidation benefits. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanone, methoxy flavanones, unsubstituted chalcone, 2′,4-dihydroxy chalcone, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones such as soy isoflavones, including but not limited to genistein, daidzein, and equol, their glucosyl derivatives, and mixtures thereof.

The compositions of the present invention may comprise from about 0.01% to about 20%, alternatively from about 0.1% to about 10%, and alternatively from about 0.1% to about 5% of flavonoids.

vii. Hair Growth Regulators

The compositions of the present invention may comprise compounds useful for regulating hair growth. Suitable hair growth regulators include, but are not limited to, hexamidine, butylated hydroxytoluene (BHT), hexanediol, panthenol and pantothenic acid derivates, their isomers, salts and derivatives, and mixtures thereof. The compositions of the present invention may comprise from about 0.0001% to about 20%, alternatively from about 0.001% to about 10%, alternatively from about 0.01% to about 5%, and alternatively from about 0.1% to about 2% of hair growth regulators.

viii. Other Skin Care Actives

The compositions of the present invention further may comprise non-vitamin antioxidants, preservatives, phytosterols and/or plant hormones, protease inhibitors, tyrosinase inhibitors, anti-inflammatory agents and N-acyl amino acid compounds.

Suitable non-vitamin antioxidants include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), ubiquinone (co-enzyme Q10), and combinations thereof.

Suitable examples of plant sterols and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIVHITE® from Seppic (France).

Other useful skin care actives include dehydroepiandrosterone (DHEA), its analogs and derivatives; alpha- and beta-hydroxyacids, including glycolic acid and octanoyl salicylate, arbutin, dimethyl aminoethanol (DMAE), kojic acid, dihydroxy acetone (DHA), soy proteins and peptides (for example, protease inhibitors such as soybean trypsin inhibitor, and Bowman-Birk inhibitor), arbutin, their isomers, salts, and derivatives, and mixtures thereof.

e. Other Optional Ingredients

The compositions of the present invention can contain a wide variety of additional ingredients, provided that they do not unacceptably alter the benefits of the invention. When incorporated into the composition, these ingredients should be suitable for use in contact with mammalian keratinous tissue without undue toxicity, incompatibility, instability, allergic response, and the like, within the scope of sound judgment. Examples of suitable classes include, but are not limited to, absorbants, aesthetic components such as fragrances, pigments, anti-microbial agents, colorings, skin conditioners, emollients, skin sensates, buffering agents, bulking agents, chelating agents, colorants, opacifying agents, pH adjusters, preservatives, propellants, skin bleaching and lightening agents, skin firming agents, skin soothing and/or healing agents and derivatives, and mixtures thereof. It should be noted, however, that many materials may provide more than one benefit, or operate via more than one mode of action. Therefore, classifications herein are made for the sake of convenience and are not intended to limit the active to that particular application or applications listed.

IV. Method

The present invention provides for a method of regulating and/or improving the condition of mammalian keratinous tissue, comprising the step of topically applying a first composition comprising hydrophobic modified silica, a particulate material and a dermatologically-acceptable carrier. Alternatively, the method may comprise the step of applying a second composition comprising a dermatologically acceptable acidic compound. Alternatively, the method may comprise the step of applying at least one additional composition as described herein.

The second and additional compositions of the present invention may take a variety of final forms, non-limiting examples of which include lotions, creams, pastes, milks, liquids, gels, solid forms, sprays, mousses and eye jellies. In one embodiment, the second and additional compositions are in the form of a liquid or a cream suitable for application to the face, neck and other exposed areas of the body. The second and additional compositions also may be releasably applied to a substrate, and may be used in combination with a delivery enhancement device, non-limiting examples of which include an implement, a spray applicator, a brush, automated scrubbing device, an ultrasound device, and combinations thereof. In one embodiment, the second and/or additional composition is applied with a spray applicator.

A. Second Composition

When the method of the present invention comprises application of a second composition comprising a dermatologically acceptable acidic compound, the second composition is applied so as to contact, or commingle with, the first composition. The second composition further may comprise any of the ingredients and skin care actives described herein. When the second composition is applied to a first composition comprising a bicarbonate salt, effervesce may occur. The second composition may have a pH of from about 1 to about 6, alternatively from about 2 to about 5, and alternatively from about 2.5 to about 4.5.

Suitable dermatologically acceptable acidic compounds include, but are not limited to, alpha- and beta-hydroxy acids, their salts, isomers and derivatives, and mixtures thereof. Non-limiting examples of alpha- and beta-hydroxy acids include alpha-hydroxy-butyric acid, alpha-hydroxyisobutyric acid, alpha-hydroxyisocaproic acid, alpha-hydroxyisovaleric, atrolactic acid, beta-hydroxybutyric acid, beta-phenyl lactic acid, beta-phenylpyruvic acid, citric acid, ethyl pyruvate, galacturonic acid, glucoheptonic acid, glucoheptono 1,4-lactone, gluconic acid, gluconolactone glucuronic acid, glucuronolactone, glycolic acid, isopropyl pyruvate, lactic acid, malic acid, mandelic acid, ethyl pyruvate, mucic acid, pyruvic acid, saccharic acid, saccharic acid 1,4-lactone, salicylic acid, tartaric acid, tartronic acid, and mixtures thereof. In one embodiment, the acidic compound is lactic acid.

The second composition may comprise from about 0.1% to about 25%, alternatively from about 1% to about 20%, and alternatively from about 2% to about 15% of the acidic compound.

B. Additional Compositions

The method of the present invention may comprise application of one or more additional compositions to the keratinous tissue. The additional compositions may comprise any of the ingredients and skin care actives described herein.

C. Emulsion

The second and additional compositions of the present invention may be in the form of an emulsion, non-limiting examples of which include oil-in-water, water-in-oil, silicon-in-water, silicone-in-oil, and mixtures thereof. Emulsions may contain a humectant, for example, glycerin. Emulsions further may comprise an emulsifier. Emulsifiers may be nonionic, anionic or cationic. Non-limiting examples of suitable emulsifiers are disclosed in U.S. Pat. No. 3,755,560 issued to Dickert et al., U.S. Pat. No. 4,421,769, issued to Dixon et al., and McCutcheon's Detergents and Emulsifiers, North American Edition, pages 317-324 (1986). Suitable emulsions may have a wide range of viscosities, depending on the desired product form.

D. Application of the Compositions

Application of any of the compositions can occur through a variety of means, including with the fingers or hands, by using an implement or other delivery enhancement device, and combinations thereof. Non-limiting examples of implements include a pad, cotton ball, applicator pen, spray applicator, substrates and patches.

A wide range of quantities of any of the compositions of the present invention can be employed to improve the condition of the skin. Quantities of the present compositions typically applied per cm² of skin are from about 0.1 mg/cm² to about 20 mg/cm². Alternatively, a suitable application amount is about 0.5 mg/cm² to about 10 mg/cm². The composition may be applied to any part of the external portion of keratinous tissue, including face, under-eye area, eyelids, scalp, neck, torso, arms, underarms, hands, legs, feet, eyelashes, eyebrows, and combinations thereof. Alternatively, the composition is delivered to the face and/or neck.

E. Application Regimens

The composition of the present invention may be applied to the keratinous tissue in combination with a second composition and/or at least one additional composition. When part of an application regimen involving more than one composition, the composition comprising hydrophobic modified silica, a particulate material and a dermatologically-acceptable carrier may be referred to as “the first composition.” The term “second” is not intended to imply a temporal aspect; the second and/or any additional compositions may be applied prior to and/or after application of the first composition.

After application, the first, second and/or additional compositions may be allowed to remain on the skin for a period of time prior to being removed. The period of time will be sufficient to achieve the desired benefit without resulting in any undue side effects. In one embodiment, the composition(s) may be allowed to remain on the skin overnight, alternatively for 30 minutes, alternatively for 5 minutes, and alternatively for 1 minute. In one embodiment, the first composition may be gently rubbed onto the skin by one's fingertips and/or with an implement.

Removal of any of the compositions may occur through a variety of means, for example, rinsing and/or wiping. In one embodiment, the composition(s) are rinsed from the skin with water.

In one embodiment, the first composition may be applied to an area of skin, and allowed to remain on the skin, for example, for 30 minutes. The composition may be gently rubbed into the skin with the fingers, an implement, or delivery enhancement device to enhance, for example, the exfoliation benefits. The composition may then be rinsed from the face with water and/or with a composition comprising water. If the composition is substantially anhydrous, a warming sensation may be produced when water is applied to the composition. Alternatively, if the composition comprises a sulfate salt and/or a chloride salt, for example, magnesium sulfate and/or calcium chloride, a warming sensation may be produced upon rinsing.

In yet another embodiment, the first composition may be applied as described above and allowed to remain on the skin for a suitable length of time. The second composition containing an acidic compound may then be applied to the face and allowed to remain on the skin, for example, for 1 minute. The compositions are rinsed from the face with water and/or with a composition comprising water. If the first composition contains a bicarbonate salt, effervescence may occur.

In yet another embodiment, the first and second compositions may be applied as described above. After rinsing, an additional composition may be applied to, for example, moisturize and protect the skin. The additional composition may be allowed to remain on the skin.

In yet another embodiment, prior to applying the first composition, an additional composition may be applied to the skin to, for example, cleanse and/or tone the skin. The cleansing/toning composition may be rinsed from the skin, and the first, second and/or additional compositions may be applied to the skin as described above.

V. Kit

The present invention further may comprise a kit, said kit comprising first composition and a second composition as described herein. The kit further may comprise one or more additional compositions, instructions for applying the composition(s), instructions for complying with a suitable application regimen, an implement, a substrate, a delivery enhancement device, a dietary supplement, and combinations thereof. The kit may comprise an outer packaging unit, which in turn may comprise one or more smaller, inner packaging units. The inner packaging units may comprise one or more of the individual components of the kit. The inner and outer packaging units may be of any type suitable for containing, presenting and/or reasonably protecting from damage the contents of the kit. The inner packaging units each may contain a quantity of a composition suitable for use in a single application regimen. In one example, the individual packaging units each will contain 10 ml, alternatively 5 ml, alternatively 2 ml, and alternatively 1 ml of a composition described herein.

### EXAMPLES

| | I | II | III | IV | V |
|---|---|---|---|---|---|
| | % | % | % | % | % |
| INGREDIENTS | w/w | w/w | w/w | w/w | w/w |
| PEG-4 | — | QS | — | — | — |
| PEG-8 | QS | — | QS | QS | — |
| PROPYLENE GLYCOL | — | — | 5 | — | QS |
| DIPROPYLENE GLYCOL | — | — | 10 | — | — |
| PENTYLENE GLYCOL | — | — | 5 | — | — |
| GLYCERIN | — | — | — | 10 | — |
| WATER | — | — | — | 5 | — |
| SODIUM BICARBONATE, | 34 | 15 | — | 20 | 25 |
| COARSE (GRADE 5) | | | | | |
| SODIUM BICARBONATE, | — | 15 | — | — | 10 |
| FINE (GRADE 1) | | | | | |
| MAGNESIUM SULFATE | — | — | 15 | — | — |
| DRYFLO PLUS¹ | — | — | — | 2 | — |
| MICROTHENE FN510-00² | — | — | 2 | — | — |
| TITANIUM DIOXIDE | — | — | — | 0.5 | — |
| DISPERSION³ | | | | | |
| AEROSIL R812 | 5 | — | 6.5 | 5 | — |
| CAB-O-SIL TS-530 | — | 4 | — | — | — |
| CAB-O-SIL TS-720 | — | — | — | — | 3 |
| NIACINAMIDE | 0.1 | — | — | — | 0.1 |
| D-PANTHENOL | — | 0.5 | — | — | — |
| VITAMIN C | 0.001 | — | — | — | — |
| VITAMIN E ACETATE | 0.01 | — | — | — | — |
| POLYSORBATE 20 | 4 | — | 6 | — | — |
| LAURETH-4 | — | — | — | 0.5 | — |
| METHYLPARABEN | — | — | — | 0.05 | — |
| FD&C DYES | 0.0011 | — | — | — | — |
| FRAGRANCE | 0.15 | — | — | — | — |

¹Aluminum starch octenylsuccinate powder supplied by National Starch, Bridgewater, NJ, USA
²Polyethylene powder supplied Equistar Chemicals, Houston, TX, USA
³Pigment grade titanium dioxide (75%) aqueous dispersion also containing glycerine and polyacrylate supplied by Kobo Products, Inc, South Plainfield, NJ, USA

The above compositions are prepared by combining all ingredients in a suitable container, and then mixing the composition until the composition is uniform via known mixing methods.

| | VI | VII | VIII | IX | X |
|---|---|---|---|---|---|
| | % | % | % | % | % |
| INGREDIENTS | w/w | w/w | w/w | w/w | w/w |
| PEG-4 | 30 | QS | — | — | QS |
| PEG-8 | QS | — | — | QS | — |
| PROPYLENE GLYCOL | — | — | 10 | — | — |
| PENTYLENE GLYCOL | — | — | QS | — | — |
| GLYCERIN | — | — | — | 35 | — |
| WATER | 2 | — | — | — | — |
| SODIUM BICARBONATE, | — | — | 5 | 20 | — |
| COARSE (GRADE 5) | | | | | |
| MAGNESIUM SULFATE | — | — | 5 | — | 15 |
| CALCIUM CHLORIDE | — | 10 | — | — | — |
| MICROTHENE FN510-00 | — | — | 5 | 1 | — |
| AC POLYETHYLENE 395¹ | 10 | — | 5 | — | 5 |
| PRESTIGE SILK ORANGE | — | 2 | — | — | — |
| INTERFERENCE | | | | | |
| PIGMENT² | | | | | |
| NYLONPOLY WL 10³ | 4 | — | — | — | — |
| AEROSIL R972 | — | — | — | 6 | — |
| CAB-O-SIL TS-530 | 2 | — | — | — | 4 |
| CAB-O-SIL TS-720 | 3 | 5 | 5 | — | — |
| CAB-O-SIL HS-5 | — | 0.5 | — | — | — |
| GLYCERYL | — | — | — | — | 8 |
| MONOSTEARATE | | | | | |
| NIACINAMIDE | 0.5 | — | — | — | — |
| D-PANTHENOL | 0.5 | — | — | — | — |
| VITAMIN C | — | — | 0.005 | — | — |
| VITAMIN E ACETATE | — | 0.2 | — | — | — |
| PPG-3 MYRISTYL ETHER | — | — | 3 | — | — |
| POLYSORBATE 20 | 0.5 | — | — | 1 | 4 |

¹Oxidized polyethylene powder supplied by Honeywell Specialty Chemicals, Morristown, NJ, USA
²Titanium dioxide and tin oxide coated mica supplied by Eckart, Louisville, KY USA
³Nylon-12 particles supplied by C.I.T. Sarl, France

The above compositions VI to IX are prepared by combining all ingredients in a suitable container, and then mixing the composition until the composition is uniform via known mixing methods. Composition X is prepared by combining all ingredients in a suitable container, heating to about 75° C. while mixing, and then cooling with mixing to about 40° C. and then pouring into an appropriate container.

### Example XI

Composition Example I above is applied to the skin and massaged into the skin for about 1 minute, followed by application of a second, aqueous solution comprising lactic acid, with a pH adjusted to about 3.8 through the use of sodium hydroxide. Upon application of the second solution, heat is generated, and foam is produced. After massaging the lactic acid solution into the skin for about 1 minute, both compositions are rinsed from the skin with water.

### Example XII

Composition Example III above is applied to the skin and massaged into the skin for about 1 minute, followed by application of an oil-in-water emulsion based anti-aging cream such as Example 1 from U.S. Pat. No. 5,997,887. Heat is generated upon application of the emulsion. Both compositions are rinsed from the skin with water.

### Example XIV

Composition Example VII above is applied to the skin and massaged into the skin for about 1 minute, followed by application of water-in-silicone emulsion based anti-aging product, such as Example 1 from US 2002/0022040. Both compositions are rinsed from the skin with water.

### Example XIV

Composition Example VII above is applied to the skin and massaged into the skin for about 1 minute, followed by application of an oil-in-water emulsion based sunscreen product, such as Example 1 from U.S. Pat. No. 5,759,524. Both compositions are later rinsed from the skin with water.

### Example XV

Composition Example III above is applied to the hair and massaged into the hair and scalp for about 1 minute. The composition is rinsed from the hair with water and heat is produced. A conditioning compound may then be applied to and subsequently rinsed from the hair.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) hydrophobic modified silica;
b) a particulate material;
c) a dermatologically-acceptable carrier, said carrier comprising about 70% or greater by weight of the carrier of one or more water-soluble polyols, wherein said water-soluble polyol has a solubility parameter of from about 11 to about 17.

2. The personal care composition of claim 1, wherein said particulate material is water-soluble.

3. The personal care composition of claim 2, wherein said particulate material is an inorganic salt.

4. The personal care composition of claim 3, wherein said particulate material is selected from the group consisting of bicarbonate salts, sulfate salts, chloride salts, and mixtures thereof.

5. The personal care composition claim 4, wherein said particulate material is selected from the group consisting of sodium bicarbonate, calcium chloride, magnesium sulfate, and mixtures thereof.

6. The personal care composition of claim 5, wherein said particulate material is sodium bicarbonate.

7. The personal care composition of claim 1, wherein said hydrophobic modified silica is selected from the group consisting of silica dimethyl silylate, silica silylate, silica dimethicone silylate, and mixtures thereof.

8. The personal care composition of claim 7, wherein said hydrophobic modified silica is selected from the group consisting of silica silylate, silica dimethicone silylate and mixtures thereof.

9. The personal care composition of claim 1, wherein said water-soluble polyol is selected from the group consisting of glycerol, propylene glycol, dipropylene glycol, tri-propylene glycol, butylene glycol, pentylene glycol, hexylene glycol, polyethylene glycols, and mixtures thereof.

10. The personal care composition of claim 9, wherein said water-soluble polyol is one or more polyethylene glycols.

11. The personal care composition of claim 1, wherein said dermatologically-acceptable carrier comprises about 10% or less of water.

12. The personal care composition of claim 11, wherein said dermatologically-acceptable carrier is substantially anhydrous.

13. The personal care composition of claim 1, wherein said composition further comprises at least one skin care active.

14. The personal care composition of claim 13, wherein said skin care active is selected from the group consisting of niacinamide, vitamin E, vitamin C, peptides, N-acetyl-D-glucosamine, and derivatives and mixtures thereof.

15. The personal care composition of claim 1, wherein said composition is releasably applied to a substrate.

16. A stable personal care composition comprising:
a) from about 1% to about 15% of hydrophobic modified silica;
b) from about 1% to about 50% of a particulate material selected from the groups consisting of bicarbonate salts, sulfate salts, chloride salts, and mixtures thereof;
c) from about 35% to about 98% of a dermatologically-acceptable carrier, said carrier comprising about 70% or greater, by weight of the carrier, of one or more polyethylene glycols, wherein said polyethylene glycol has a solubility parameter of from about 11 to about 17.

17. The personal care composition of claim 16, wherein said particulate material is selected from the group consisting of sodium bicarbonate, magnesium sulfate, calcium chloride, and mixtures thereof.

18. The personal care composition of claim 17, wherein said particulate material is sodium bicarbonate.

19. The personal care composition of claim 16, wherein said hydrophobic modified silica is selected from the group consisting of silica silylate, silica dimethicone silylate and mixtures thereof.

20. The personal care composition of claim 16, wherein said carrier is substantially anhydrous.

21. The personal care composition of claim 16, wherein said composition further comprises at least one skin care active selected from the group consisting of niacinamide, vitamin E, vitamin C, peptides, N-acetyl-D-glucosamine, and derivatives and mixtures thereof.

22. The personal care composition of claim 16, wherein said composition is releasably applied to a substrate.

23. A method for regulating the condition of mammalian keratinous tissue, comprising the step of applying a first composition to the keratinous tissue, said first composition comprising:
a) hydrophobic modified silica;
b) a particulate material;
c) a dermatologically-acceptable carrier, said carrier comprising about 70% or greater by weight of the carrier of one or more water-soluble polyols, wherein said water-soluble polyol has a solubility parameter of from about 11 to about 17.

24. The method of claim 23, wherein said particulate material is selected from the group consisting of sulfate salts, chloride salts, and mixtures thereof.

25. The method of claim 24, wherein said particulate material is selected from the group consisting of calcium chloride, magnesium sulfate, and combinations thereof.

26. The method of claim 23, wherein said particulate material is a bicarbonate salt.

27. The method of claim 26, wherein said particulate material is sodium bicarbonate.

28. The method of claim 23, wherein said first composition further comprises at least one skin care active.

29. The method of claim 23, wherein said carrier is substantially anhydrous.

30. The method of claim 25, further comprising the step of applying water to said keratinous tissue, whereupon heat is generated.

31. The method of claim 29, further comprising the step of applying water to said keratinous tissue, whereupon heat is generated.

32. The method of claim 23, further comprising the step of applying a second composition in contact with the first composition, said second composition comprising a dermatologically acceptable acidic compound.

33. The method of claim 32, wherein said dermatologically acceptable acidic compound is a hydroxy-acid.

34. The method of claim 33, wherein said dermatologically acceptable acidic compound is lactic acid.

35. The method of claim 32, wherein said second composition further comprises at least one skin care active.

36. The method of claim 32, whereupon application of said second composition results in effervescence.

37. The method of claim 23, further comprising the step of applying one or more additional skin care compositions to the keratinous tissue.

38. The method of claim 32, further comprising the step of applying one or more additional skin care compositions to the keratinous tissue.

39. A kit comprising a stable first composition and a second composition, wherein said first composition comprises:
a) hydrophobic modified silica;
b) a particulate material;
c) a dermatologically-acceptable carrier, said carrier comprising about 70% or greater by weight of the carrier of one or more water-soluble polyols; said water-soluble polyol having a solubility parameter of from about 11 to about 17; and
wherein said second composition comprises a dermatologically acceptable acidic compound.

40. The kit of claim 39, wherein said particulate material is selected from the group consisting of bicarbonate salts, sulfate salts, chloride salts, and mixtures thereof.

41. The kit of claim 40, wherein said particulate material is selected from the group consisting of sodium bicarbonate, magnesium sulfate, calcium chloride, and mixtures thereof.

42. The kit of claim 39, wherein said acidic compound is lactic acid.

43. The kit of claim 39, said kit further comprising at least one additional skin care composition.

44. The kit of claim 39, said kit further comprising at least one dietary supplement.

45. The kit of claim 39, said kit further comprising a delivery enhancement device.

46. The kit of claim 39, said kit further comprising a substrate.
